(19) United States
(12) Patent Application Publication
(10) Pub. No.: US 2013/0316382 A1
(43) Pub. Date: Nov. 28, 2013

(54) METHOD FOR EVALUATING THE VITAL PROGNOSIS OF A SUBJECT IN A CRITICAL CONDITION

(71) Applicants: Fatima Smih, Toulouse (FR); Franck Desmoulin, Toulouse (FR); Philippe Rouet, Toulouse (FR)

(72) Inventors: Fatima Smih, Toulouse (FR); Franck Desmoulin, Toulouse (FR); Philippe Rouet, Toulouse (FR)

(73) Assignee: Institut National de la Sante et de la Recherche Medicale (INSERM), Paris (FR)

(21) Appl. No.: 13/990,454

(22) PCT Filed: Nov. 30, 2011
(86) PCT No.: PCT/EP11/71384

§ 371 Date: Aug. 13, 2013

(30) Foreign Application Priority Data
Nov. 30, 2010 (EP) 10306322.8

(51) Int. Cl. G01N 33/92

(52) U.S. Cl. CPC G01N 33/92
USPC 435/11

(57) ABSTRACT

The invention relates to a method for evaluating the vital prognosis of a subject suffering from heart failure, said method comprising the step of determining the lactate/cholesterol ratio in a biological sample of said subject and comparing the lactate/cholesterol ratio to a threshold value.

### FIELD OF THE INVENTION

The present invention relates to a method for evaluating the vital prognosis of a subject in a critical condition, for example suffering from an acute heart failure.

### BACKGROUND OF THE INVENTION

Acute heart failure (AHF) is the most common cause of hospital admission among patients above 65 years old and a common presentation of seriously ill patients admitted to an intensive care unit. A survey on the quality of care among patients with heart failure in Europe has shown a mortality rate of 13.5% between admission of the subject in hospital and 12 weeks follow-up.

Nowadays, evaluation of heart failure patients includes a focused history, physical examination, an electrocardiogram and an echocardiogram. Altogether, these complementary approaches are aimed at better management strategies. Measurements of the biomarker brain natriuretic peptides (BNPs) are the most commonly used HF biomarkers associated with altered hemodynamics. BNPs are of both diagnostic and prognostic importance. Recently, BNPs levels at admission were shown to be significantly higher in patients that suffered a cardiac death within 90 days of hospital discharge. However, blood BNPs level monitoring has limitations and rawbacks. Indeed, several reports have underlined their high variability despite some improvement gained with the quantification of amino-terminal pro-BNP. The BNPs level monitoring was shown to be not significative when said level is comprised between 100 and 300 pg/ml.

The quest for prognosis biomarkers is an ongoing challenge because so far no blood molecule identified meets all the requirements of a perfect biomarker: high level of reliability and cost effectiveness. Thus, there is a need for identification of diagnostic molecules that would provide an improvement over existing biomarkers for vital prognosis of a subject in a life-threatening condition, such as acute heart failure. There is thus a need for a simple test to identify patients with a higher mortality risk in order to optimize medical care.

### SUMMARY OF THE INVENTION

Low plasma cholesterol levels have been associated with increased in-hospital mortality of patients with various diseases, including miscellaneous heart diseases, and were proposed as one of the first signs of forthcoming deterioration of a preexisting disease. Indeed, hypocholesterolemia is a signal of disease state in a number of pathologies. These include hepatic failure, hypertyroidism, malnutrition, poor digestive absorption, inflammatory syndromes, trauma and infectious diseases. More recently, hypocholesterolemia was associated with high peri-operative mortality in patients supported by a left ventricular assistant system and with increased mortality, for example in acute heart failure patients.

The inventors have shown that lactate and cholesterol concentrations were highly accurate and appropriate for determining the prognosis of surviving of a subject in a critical condition, for example being hospitalised in emergency or admitted to an intensive care unit and in need for a efficient medical care. The inventors have evidenced that venous lactate and cholesterol levels, with the help of their ratio, may define a severity index of the heart failure of a subject in a critical condition. This value is proportional to the severity of the condition of said subject, which renders said ratio highly appropriate for helping the physician to choose and adapt a treatment strategy for the subject in need thereof. The inventors have further shown that said ratio was independent of BNP level, which displayed a poor predictive value in in-hospital mortality study.

The invention thus provides a method for evaluating the vital prognosis of a subject in a critical condition, for example suffering from a life-threatening disease such as acute heart failure, said method comprising the step of determining the lactate/cholesterol ratio in a biological sample of said subject.

### DETAILED DESCRIPTION OF THE INVENTION

The invention provides a method for evaluating the vital prognosis of a subject in a critical condition, preferably suffering from acute heart failure, said method comprising the step of determining the lactate/cholesterol ratio in a biological sample of said subject.

The expression “vital prognosis” as used herein refers to predicting the course or outcome of a critical and life-threatening condition in a subject. This does not refer to the ability to predict the course or outcome of a condition with 100% accuracy, or even that a given course or outcome is predictably more or less likely to occur based on the pattern of biomarkers. Instead, the person skilled in the art will understand that the expression “vital prognosis” refers to an increased probability that a certain course or outcome will occur.

The term “subject” or “patient” denotes a human in a critical condition for which an indication of the health outcome is needed. Preferably said subject is in a critical condition and may be hospitalised in emergency. Most preferably, said subject suffers from an acute heart failure.

The term “healthy individual” denotes a human which is known to be healthy, i.e. which is not in a critical condition and does not need any medical care. Preferably, said healthy individual does not suffer from acute heart failure and/or has never been subject to such acute heart failure.

As used herein, “critical condition” refers to a condition of a subject for which the vital signs are unstable and/or diminished. This term refers to a serious and life-threatening condition, usually following a serious trauma. Typically, a subject in a critical condition may exhibit various symptoms and signs such as:
signs of hypovolaemic shock such as anxiety, restlessness, altered mental state, hypotension, tachycardia, hypothermia, dilated pupils; and/or
signs of cardiogenic shock such as distended jugular veins, weak or absent pulse, arrhythmia, and tachycardia; and/or
signs of obstructive shock such as distended jugular veins and pulsus paradoxus; and/or
signs of septic shock such as pyrexia, systemic vasodilation resulting in hypotension, reduced contractility of the heart, disseminated intravascular coagulation, or increased levels of neutrophils; and/or
signs of neurogenic shock such as bradycardia, or priapism due to peripheral nervous system stimulation; and/or
signs of anaphylactic shock such as skin eruptions and large bumps, localised oedema, weak and rapid pulse, or breathlessness and cough.

Assessing the aforementioned signs is important for the physician to identify subject suffering from a life threatening condition. Indeed, the early identification and rapid treatment of a subject showing signs of a critical and serious condition is widely acknowledged as a vital step towards improving survival. Typically, a subject presenting the above mentioned signs may suffer from various conditions such as a heart failure, sepsis condition especially septic shock, acute breath decompensation, or cardiac arrest.

As used herein “heart failure” refers to a condition that occurs when a problem with the structure or function of the heart impairs its ability to supply sufficient blood flow to meet the body's needs. This term encompasses chronic heart failure, acute heart failure, myocardial infarction, unstable angina, cardiac thrombus, diastolic dysfunction, systolic dysfunction.

The term “chronic heart failure” as used herein means a case of heart failure those progresses so slowly that various compensatory mechanisms work to bring the disease into equilibrium.

“Acute heart failure” or “acute chronic heart failure” as used herein refers to both sudden onset heart failure, as well as acute “exacerbated” or “decompensated” heart failure, referring to episodes in which a patient with known chronic heart failure or devoid of chronic heart failure abruptly develops worsening symptoms and requires hospitalization. Common symptoms of complications due to acute heart failure include, but are not limited to, dyspnea due to pulmonary congestion or cardiogenic shock due to low cardiac output, easy fatigueability (exercise intolerance), peripheral edema, anasarca (pronounced generalized edema), nocturia (frequent nighttime urination), bradycardia, heart block, hypotension, dizziness, syncope, diabetes, oliguria or anuria, hypokalemia, bronchospasm, cold sweat, and asthma.

“Myocardial infarction” as used herein refers to a condition in which necrotic changes in the myocardium or heart muscle that results from obstruction of an end artery.

“Unstable angina”, as used herein refers to a condition in which the heart does not get enough blood flow and oxygen. Such condition is referred to as a prelude to a heart attack.

“Cardiac thrombus” as used herein refers to the formation of a blood clot in the cardiac tissue.

“Diastolic dysfunction” refers to an abnormality in the heart's (i.e., left ventricle's) filling during diastole. Diastole is the phase of the cardiac cycle when the heart (i.e. ventricle) is not contracting but is relaxed and filling with blood that is being returned to it (either from the body (into right ventricle) or from the lungs (into left ventricle). Typically, diastolic dysfunction denotes a stiffer ventricular wall, which leads to inadequate filling of the ventricle, and therefore an inadequate stroke volume. The failure of ventricular relaxation also results in elevated end-diastolic pressures. Diastolic dysfunction may not manifest itself except in physiologic extremes if systolic function is preserved. The patient may be completely asymptomatic at rest, but is extremely sensitive to increases in heart rate and sudden bouts of tachycardia.

“Systolic dysfunction” refers to an abnormality in the heart's (i.e., left ventricle's) ability to pump blood out of the chamber into the systemic circulation. Systole is a phase of the cardiac cycle where the myocardium is contracting in a coordinated manner in response to an endogenous electrical stimulus, and pressure is being generated within the chambers of the heart driving blood flow. Experimental and clinical measurements of systolic contraction are often based on ejection fraction and cardiac output.

As used herein, the term “biological sample” as used herein refers to any biological sample of a subject. Preferably, said sample is a body fluid of said subject. Non-limiting examples of samples include, but are not limited to, blood, serum, plasma, nipple aspirate fluid, urine, saliva, synovial fluid and cephalorachidian liquid (CRL). Preferably, the biological sample is a blood sample, preferably venous blood.

As used herein, the term “cholesterol” and the term “total cholesterol” may be used interchangeably. The expression “total cholesterol” refers to the global amount of cholesterol present in plasma which includes the free, esterified and peptide-bound cholesterol that is contained in the major plasma lipoproteins such as chylomicrons, VLDL, LDL and HDL.

The term “free” as used herein refers the physicochemical property of a molecule in solution which can be free by opposition to bounded to other molecules as peptide giving “peptide-bound” molecular complexes.

The term “esterified” as used herein refers to capability of cholesterol to form a cholesteryl ester with most of fatty acids.

The term “lipoproteins” as used herein refers to protein spheres that transport cholesterol, triglyceride, or other lipid molecules through the bloodstream. Lipoproteins are categorized into five types according to size and density.

The term “chylomicron” as used herein refers to the largest in size and lowest in density of the triglyceride carrying lipoproteins.

As used herein, “HDL” or “high-density lipoprotein” relates to a class of plasma lipoprotein with a high proportion of protein, including apolipoproteins A, C, D and E. HDL incorporates and transports cholesterol, whether free or esterified, in the plasma as an HDL-cholesterol complex.

As used herein, “LDL” or “low-density lipoprotein” relates to a class of plasma lipoprotein with a high proportion of lipid, including cholesterol, cholesterol esters and triglycerides. It includes primarily apolipoprotein B-100 and apolipoprotein E. LDL incorporates and transports cholesterol in the plasma.

The term “VLDL” or “very low density lipoprotein” as used herein refers to a triglyceride carrying lipoprotein.

Preferably, said subject suffers from heart failure such as chronic heart failure, acute heart failure, myocardial infarction, unstable angina, cardiac thrombus, diastolic dysfunction, systolic dysfunction. Alternatively, said subject has a decreased left ventricular systolic function. Most preferably, said subject suffers from an acute heart failure.

“Left Ventricular Ejection Fraction” or “LVEF” refers to a measure of systolic function of the left ventricle. The ejection fraction is the percentage of blood ejected from the left ventricle with each heart beat. A reduced LVEF, for example, less than or equal to about 45% indicates that a cardiomyopathy is present.

In one particular embodiment, said subject is also reported to have significant comorbid conditions, including but not limited to hypertension, coronary heart disease, and diabetes mellitus.

For determining the lactate/cholesterol ratio, the person skilled in the art may determine the concentrations of cholesterol and lactate in a biological sample of a subject in a critical condition. Typically, determination of the concentrations of cholesterol and lactate may be performed according to routine methods well known by the skilled person in the art by using spectrophotometry such methods includes, but are not limited to, Magnetic Resonance Spectroscopy, colorimetry, fluorimetry; spectrometry with Mass spectrometer, or electrochemical methods. These methods are associated but not necessarily to enzymatic process in order to indirectly quantified the lactate and the total cholesterol such as, but not limited to, lactate deshydrogenase, lactate oxidase; cholesterol esterase, cholesterol oxidase. Alternatively, the person skilled in the art may also use common technique such as standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith.

Said measures may be expressed in the usual and commonly accepted units. For instance, they can be expressed in mg per liter of biological sample, preferably venous blood or in mmol per liter of biological sample, preferably venous blood.

Preferably, both concentrations are expressed in the same unit, most preferably in mmol per liter of blood so that the lactate/cholesterol ratio is expressed without any unit.

Non-limiting examples for assessing the concentration of cholesterol are developed in Betty Hawthorn, A comparison of some methods of cholesterol measurement, Cholesterol methods, vol. 10, no. 3, 1964. Typically, the determination of the concentration of cholesterol is performed with an enzymatic assay. Such essay may be, for instance, based on cholesterol oxidase (CHOD) and phenol-aminophenazone (PAP). This method is referred to as CHOD PAP method and is based on the determination of D4 cholestenone after enzymatic cleavage of the cholesterol ester by cholesterol esterase, conversion of cholesterol in cholesterol oxidase, and subsequent measurement by the Trinder reaction of the hydrogen peroxide formed. Preferably, in the context of the present invention, cholesterol is measured by the CHOD-PAP method with kit A11A01634 (HORIBA ABX diagnostic, Montpellier, France).

For the determination of the concentration of lactate, the one skilled in the art may use an automated lactate analyser which can measure the blood lactate concentration rapidly in a small sample of blood. Non limiting examples of such analyser are Lactate Pro™ (LP) (Arkray™, Kyoto, Japan) and Accusport™ (ACC) (Roche Diagnostics, Basel, Switzerland). Preferably, in the context of the present invention, lactate is measured by the enzymatic colorimetric Trinder method with kit A11A01721 (HORIBA ABX diagnostic).

In one embodiment, the method of the invention further comprises the step of comparing the lactate/cholesterol ratio to a threshold value.

Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art.

Preferably, the person skilled in the art may compare the lactate/cholesterol ratio obtained according to the method of the invention with a defined threshold value.

Preferably, said threshold value is the mean lactate/cholesterol ratio of a population of healthy individuals, preferably of individuals known to be healthy, i.e. which are not in a critical situation, and preferably which do not suffer from acute heart failure.

Typically, the skilled person in the art may determine the lactate/cholesterol ratio in a biological sample, preferably blood, of a statistical sample from the population of individuals known to be healthy, preferably 100 healthy individuals. The mean value of the obtained ratios is then determined, according to well known statistical analysis, so as to obtain the mean lactate/cholesterol ratio. Said value is then considered as being normal and thus constitute a threshold value.

The inventors have established a threshold value for the ratio lactate/cholesterol for easily sorting out subjects with higher risk of mortality. Indeed, by comparing the lactate/cholesterol ratio obtained in a biological sample, preferably blood, of a given subject to a threshold value, one can easily determine whether or not said subject is in a critical condition.

Preferably, the lactate/cholesterol ratio is obtained with concentrations of lactate and cholesterol expressed in mmol of liter of biological sample. Preferably, this threshold value is comprised between about 0.2 and about 0.7, preferably between about 0.3 and about 0.6, preferably between about 0.3 and about 0.5, preferably between about 0.35 and about 0.45, preferably between about 0.37 and about 0.43, preferably between about 0.39 and about 0.42, preferably between about 0.40 and about 0.42, most preferably said threshold value is about 0.41.

This threshold value is compared to a lactate/cholesterol ratio, which is obtained with concentrations of lactate and cholesterol expressed in mmol of liter of biological sample. Thus, if the one skilled in the art determines a lactate/cholesterol ratio based on lactate and cholesterol concentrations expressed in mg of liter of biological sample, the threshold value would have to be adapted. Such adaptation of the threshold value falls within the ability of the skilled person thanks to his general knowledge (which includes the molecular masses of lactate and cholesterol).

The skilled person is then able to compare the lactate/cholesterol ratio obtained in a biological sample of a subject to the mean lactate/cholesterol ratio. By using the well known techniques of statistical analysis, the skilled person in the art may then easily determine whether the difference between said ratio and threshold are statistically significant or not.

By comparing the lactate/cholesterol to this threshold value, the physician is then able to determine the vital prognosis of a subject in an emergency situation. Accordingly, the physician would be able to adapt and optimize appropriate medical care of a subject in a critical and life-threatening condition, for example suffering from acute heart failure. The determination of said prognosis is highly appropriate for follow-up care and clinical decision making. Thus, thanks to the method of the invention, the physician can easily sort out the subjects who could benefit from intensive care treatment.

Therefore, the invention is drawn to a method for evaluating the vital prognosis of a subject in a critical condition, preferably suffering from acute heart failure, said method comprising the following steps:
a) determining the lactate/cholesterol ratio in a biological sample of said subject;
b) determining the mean lactate/cholesterol ratio in a biological sample of a population of healthy individuals, preferably 100 healthy individuals; and
c) a step of comparing the ratio obtained in a) to the ratio obtained in b).

In the following, the invention will be illustrated by means of the following examples as well as the figures.

### FIGURES LEGENDS

FIG. 1: Lactate and cholesterol determined from ¹H spectra.

A: Contribution of the characteristic signal of lactate at 1.36 ppm to the spectrum of aqueous fraction of the plasma extract (Aq spectrum).
B: Contribution of the characteristic signal of cholesterol at 0.68 ppm to the spectrum of lipidic fraction of the plasma extract (Org spectrum). Values are expressed as arbitrary unit (AU).
The boxes at the left in each panel correspond to favorable outcome, whereas boxes at the right in each panel correspond to in-hospital death.
* denotes “is for student's test with p<0.05”

FIG. 2: Receiver Operating Characteristic (ROC) curve analysis.

This figure shows that the accuracy was better for lactate/Chol (AUC 0.81) than for lactate (AUC 0.76) or cholesterol (AUC 0.73) alone.
BNP concentrations were not significantly different between survivors (1104±1072, pg/ml n=98 vs 1398±1451, pg/ml n=28) and non-survivors did not show any prognostic power for in-hospital death (AUC 0.52).

FIG. 3: Kaplan-Meier curve analysis of the lactate to cholesterol ratio. Kaplan-Meier curve estimation of the survival rate of the patients according to the cholesterol to lactate ratio. 126 AHF patients were included in this analysis based on Cobas Mira+ automated enzymatic detection of venous plasma lactate and cholesterol.

Dashed line and full line are for lactate/cholesterol ratio<0.41 and >0.41, respectively.

### EXAMPLE

### Methods

### Patients

126 consecutive patients were enrolled. They were admitted with AHF to the cardiology department of Rangueil Toulouse hospital. Clinical characteristics of the patients were obtained from electronic medical records at admission and are listed in Table 1.

TABLE 1
Clinical characteristics of the patients.

| Criteria | Value |
|---|---|
| Number of subject | (n = 126) |
| Age | 69 ± 15 |
| Gender | |
| Female % | 38 |
| Cardiovascular risk factors | |
| Hypertensive % | 57 |
| Diabete % | 35 |
| Dyslipidemia % | 53 |
| Obesity % | 16 |
| Heridity CD % | 10 |
| Previous cardiac disease | |
| Coronary artery disease % | 55 |
| Valvular heart disease | 30 |
| Idiopatic dilated cardiomyopathy | 11 |
| Admission diagnosis | |
| New onset of AHF % | 40 |
| Acute decompensation of CHF % | 60 |
| Acute coronary syndrome % | 48 |
| HF with reduced ejection fraction % | 75 |
| Clinical presentation | |
| Acute decompensated HF % | 56 |
| Cardiogenic shock % | 34 |
| Pulmonary oedema or Hypertensive AHF % | 10 |
| Admission medication | |
| ACE inhibitor % | 37 |
| Angiotensin receptor blocker % | 21 |
| Beta-blocker % | 37 |
| Diuretic % | 58 |
| Aldosterone antagonist % | 20 |
| Antiplatelet agent % | 50 |
| Vitamin K antagonist % | 30 |
| Admission Labs | |
| BNP (pg/ml) | 1153 ± 1141 |
| Na+ (mM) | 136 ± 4 |
| Creatinine (μM) | 147 ± 82 |
| C reactive protein (mg/l) | 49 ± 66 |
| Hb (g/dl) | 14 ± 12 |
| Glucose (mM) | 7 ± 2 |
| Bilirubin (μM) | 20 ± 15 |
| Prothrombin ratio % | 66 ± 26 |
| LDL (g/l) | 1.06 ± 0.44 |
| HDL (g/l) | 0.45 ± 0.15 |
| TG (g/l) | 1.05 ± 0.43 |
| Admission vitals | |
| Systolic blood pressure | 120 ± 38 |
| Diastolic blood pressure | 69 ± 21 |

Heridity CD = occurrence of cardiovascular disease in first degree relatives; AHF = Acute cardiac failure; CHF = cardiac heart failure; HF = heart failure; ACE = angiotensin-converting enzyme; BNP = Brain Natriuretic Peptide; LDL = Low Desensity Lipoproteins; HDL: high Density Lipoproteins; TG = Triglycerides; BMI = body mass index; LVEF = left ventricular ejection fraction; LV = left ventricular.

The specified end point was in-hospital cardiac death which was recorded in the cardiology department within 40 days. After discharge from the hospital, the patient was re-evaluated through a review of the electronic medical record or via telephone conversation to evaluate for the survival within this period. This research protocol was approved by the institutional review boards and ethics committee. All participants gave written informed consent.

### Samples Processing

Venous blood samples were collected on the admission day in Becton Dickinson Vacutainer CPT Tube with sodium heparin. After centrifugation the plasma was collected and stored at −80° C. Immediately before analysis plasma sample was rapidly thawed at 4° C. and 250 μl as well as 1 ml aliquots were collected. A 250 μl aliquot was reconstituted into 755 μl with 500 μl of 0.9% saline in D₂O and 5 μl of 100 mM sodium 3-(trimethylsilyl) propionate-2,2,3,3-d4 (TSP) as a chemical shift reference. The 1 ml aliquot was frozen and kept at −80° C. until liquid-liquid extraction process. Simultaneous extraction of lipophilic and polar metabolites was performed with ice-cold methanol, chloroform and water (2:2:1.3, v/v/v) (13). The aqueous fraction of the extract was reconstituted in 600 μl of D₂O phosphate buffered solution with 10 μl of a 10 mM 3-(trimethylsilyl)-1-propanesulfonate sodium salt (TMPS) before NMR analysis. The organic fraction of the extract was reconstituted in 1 ml CDCl₃ with 10 μl TCB (100 mM) and maintained under nitrogen atmosphere at −80° C. until NMR analysis.

### Metabonomic Analysis

Acquisition of ¹H NMR spectra.

¹H NMR spectra were recorded at 300K on a Bruker Avance DRX 600 spectrometer operating at 600.13 MHz and equipped with a 5 mm triple axis inverse (TXI) gradient cryoprobe. Two diluted plasma spectra were sequentially acquired with presaturation of the water signal by using one-pulse (Zg spectrum) and CPMG spin-echo sequence (cpmg spectrum) with an echo loop time (2nπ) of 320 ms. A total of 64 transients were sampled with a spectral width of 12 ppm, 32 K data point on time domain (2.3 s acquisition time) and 2.5 s additional relaxation delay. Spectra of aqueous and organic fractions were serially acquired using an automatic sample changer (B-ACS 60). Spectra of aqueous fractions (Aq. spectrum) were obtained with similar parameters to the one-pulse spectrum of the diluted plasma whereas spectra of the organic fraction (Org. spectrum) were acquired with an additional delay of 4 s and without solvent suppression. ¹H NMR spectra were processes using the TOPSPIN (version 2.1, Bruker BioSpin SA, France) and AMIX (Bruker Analytik, Rheinstetten, Germany) software packages. Typical processing parameters were 65 K zero-filling and an exponential apodizing function (0.3 Hz) applied prior to Fourier transform. Phase and base-line corrections of spectra were performed by operator and referenced with AMIX software to methyl resonance of TMPS, lactate or TCB for diluted plasma, aqueous fraction and organic fraction respectively.

### Data Reduction and Pattern Recognition

Spectra of diluted plasma were bucketed from 8.25 to 0.50 ppm into 388 equal-width bins of 0.02 ppm and normalized to unit sum. These generated variables were identified with the central chemical shift value of the bins and Zg. or cpmg. as prefixes. The regions 6.00-5.39 ppm; 5.10-3.36; 2.29-2.19; 1.7-1.54; 1.17-1.14; 0.93-0.91; 0.88-0.86 were removed to eliminate baseline effects of imperfect water saturation and signals which have been ascribed to additives from CPT tubes. Spectra of the aqueous fraction were bucketed first from 5.30 to 0.30 into 127 equal-width bins of 0.04 ppm and normalized to unit sum. These generated variables were identified with the central chemical shift value of the bins and Aq. as prefix. Aromatic regions of the aqueous fraction spectra were bucketed separately from 7.47 to 6.75 ppm into 72 equal-width bins of 0.04 ppm and normalized to unit sum. These generated variables were identified with the central chemical shift value of the bins and Arom. as prefix. Finally spectra of the organic fraction were bucketed from 6.5 to 0.25 ppm into 157 equal-width bins of 0.04 ppm and normalized to unit sum; these generated variables were identified with the central chemical shift value of the bins and Org. as prefix. The regions 2.72-1.40 and 4.2-3.2 ppm were removed to eliminate signals from residual water and shifting signals respectively. These data series were exported into the SIMCA-P+ (version 12.0, Umetrics, Umeå, Sweden) software to be separately orthogonalized with an OSC filtering function (15) prior fusionned in a normalized matrix of 673 rows (X-variables) and 123 lines. Score plot of the Principal Component Analysis (PCA) allowed the identification of outliers which were outside the confidence level of 95%. Thus, out of 126 patients analyzed 4 patients from the group of survivors were rejected. To maximize separation between the groups, partial least squares-discriminant analysis (PLS-DA) was performed by using favorable outcome or in-hospital mortality as Y variables.

### Automated Lactate and Cholesterol Levels Targeted Monitoring and BNP Analysis

Lactate and total cholesterol concentration of venous blood were further determined by routine laboratory methods using a COBAS MIRA+ autoanalyzer according to the manufacturer's instructions (HORIBA ABX diagnostic, Montpellier, France). Cholesterol was measured by the CHOD-PAP method with kit A11A01634 and lactate was measured by the enzymatic colorimetric Trinder method with kit A11A01721 (HORIBA ABX diagnostic, Montpellier, France). BNP was assessed using a Centaur Bayer kit (Bayer HealthCare, France).

### Statistical Analysis

Multivariate data analysis was performed using SimcaP+ software (12.0.1) (Umetrics). Continuous variables are presented as means (±SD) and categorical variables as numbers and percentages. Continuous variables were compared with the use of student's t-test or Mann-Whitney rank sum test when normality test failed and categorical variables with the use of the Pearson chi-square test (Sigma Stat). Receiver-operating characteristic (ROC) curves and Kaplan-Meier curves were constructed using Medcalc software (MedCalc Software bvba, Belgium). P values of less than 0.05 were considered to indicate statistical significance.

### Results

### Demographic Data and Clinical Parameters

The demographic characteristics of the patients are listed in Table 1. The majority of the patients were men (62%), and 75% of all patients had a decreased left ventricular systolic function, defined as a left ventricular ejection fraction≦45%.

The majority of the patients were also previously diagnosed with significant comorbid conditions, including hypertension (57%) coronary heart disease (55%) and diabetes mellitus (35%). Of these 126 patients with severe AHF, 28 (22%) died during hospitalization. No patient died after the discharge.

### NMR Metabonomic Profiling of Patients' Plasmas

Out of 126 samples analyzed by ¹H NMR spectroscopy, 98 were from patients with a favorable outcome following decompensated heart failure and 28 were from patients who died during hospitalization. Metabonomic data could sort efficiently these two groups using partial least-square discriminant analysis. Discriminant variables from each group are the ones with most extreme values on the loading plot.

Metabolites whose protons gave rise to signals generating the highest discriminatory power variables are listed in Table 2.

TABLE 2
List of the 15 first NMR spectral variables which have the highest
influence parameter on projection of the PLS-DA analysis. Influence
parameter values range from 2.36 to 0.02 for the 432 variables analyzed.
Spectral data identification refer to the acquisition method and type
of plasma sample analyzed that finally consisted of 4 spectra analyzed
per individual (zg; cpmg; Aq; org) as described in material and methods.
Bucket generated during sampling of spectra is identified by its centered
chemical shift value in ppm.

| | | | CORRELATION SIGN | |
|---|---|---|---|---|
| Discriminating | | Contributing | In-hospital | |
| variable | IP | metabolites | death | Survival |
| zg_0.84 ppm | 2.05 | terminal methyl | negative | positive |
| | | groups of lipids | | |
| | | from lipoproteins | | |
| zg_1.06 ppm | 1.92 | valine leucine | negative | positive |
| | | isoleucine | | |
| zg_1.02 ppm | 1.90 | valine leucine | negative | positive |
| | | isoleucine | | |
| zg_1.04 ppm | 1.81 | valine leucine | negative | positive |
| | | isoleucine | | |
| Aq_1.08 ppm | 1.88 | valine leucine | negative | positive |
| | | isoleucine | | |
| Org_1.04 ppm | 2.36 | cholesterol | negative | positive |
| Org_0.68 ppm | 2.22 | cholesterol | negative | positive |
| Org_0.92 ppm | 2.10 | cholesterol | negative | positive |
| Org_0.96 ppm | 2.04 | cholesterol | negative | positive |
| zg_1.3 ppm | 2.16 | methylene groups | positive | negative |
| | | of lipids from | | |
| | | lipoproteins | | |
| zg_1.34 ppm | 1.98 | lactate | positive | negative |
| zg_1.32 ppm | 1.94 | methylene groups | positive | negative |
| | | of lipids from | | |
| | | lipoproteins | | |
| cpmg_1.34 ppm | 1.81 | lactate | positive | negative |
| Aq_1.36 ppm | 1.80 | lactate | positive | negative |
| Org_1.28 ppm | 2.33 | methylene groups | positive | negative |
| | | of lipids | | |

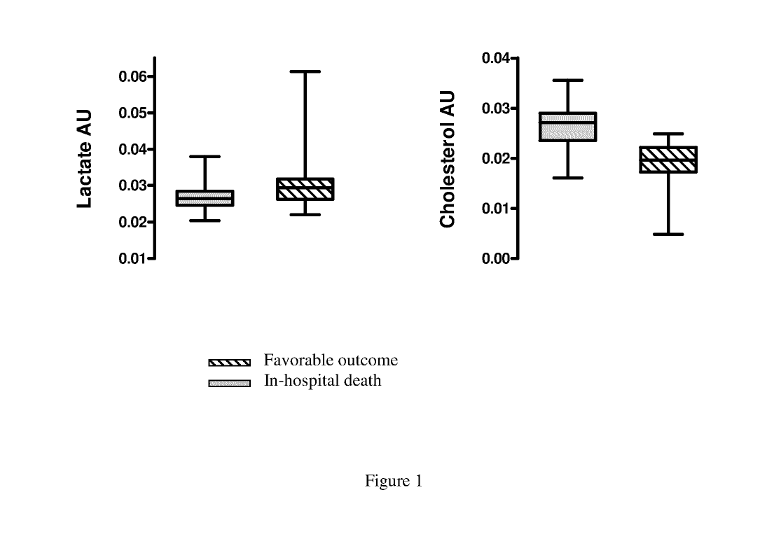

Thus, cholesterol, the branched amino-acids valine, leucine and isoleucine and terminal methyls of lipids from lipoproteins were positively correlated with survival, whereas lactate and methylene of lipids aliphatic chains were positively correlated with the in-hospital cardiac death. Conventional analysis by integration of the characteristic signal of lactate and of cholesterol from spectrum of the aqueous and lipidic fraction of the plasma confirm that contribution of lactate (FIG. 1A) and cholesterol (FIG. 1B) signals to the total spectra were significantly increased in the in-hospital death group and in the favorable outcome group, respectively.

### Automated Lactate and Cholesterol Monitoring

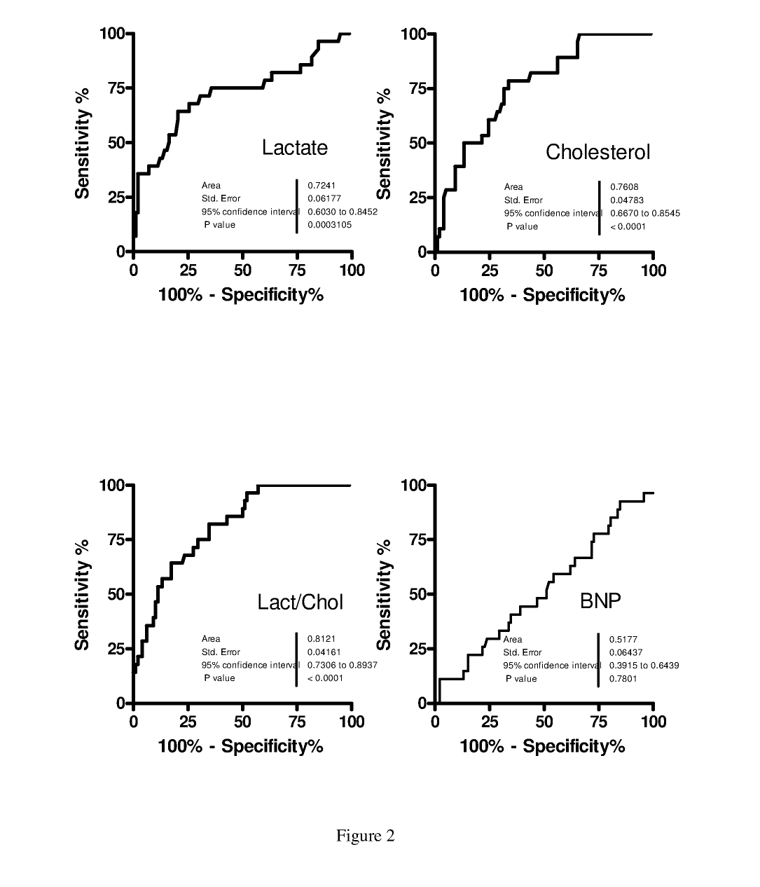

A supplementary quantitative analysis of venous plasma lactate and cholesterol were carried out by automated analysis. Lactate concentration was significantly higher in non-survivors (1.78±1.32 mM, n=28) than in survivors (1.14±0.40 mM, n=98) (p<0.001). Conversely, cholesterol concentration was significantly lower in with the non-survivors at 2.37±0.67 mM (n=28) than in the survivors at 3.23±1.01 (n=98) (p<0.001). Accuracies of lactate and cholesterol concentrations were evaluated using ROC curve analysis (FIG. 2 and Table 3).

TABLE 3
Receiver operating characteristic (ROC) curve analysis results.

| TEST | AUC | P |
|---|---|---|
| BNP | 0.52 | =0.78 |
| Cholesterol | 0.76 | <0.0001 |
| Lactate | 0.72 | <0.0003 |
| Lactate/Cholesterol ratio | 0.81 | <0.0001 |

AUC = area under the curve; P = P value for comparison with area 0.5.

BNP concentrations were not significantly different between survivors (1104±1072, pg/ml n=98 vs 1398±1451, pg/ml n=28) and non-survivors did not show any prognostic power for in-hospital death (AUC 0.52).

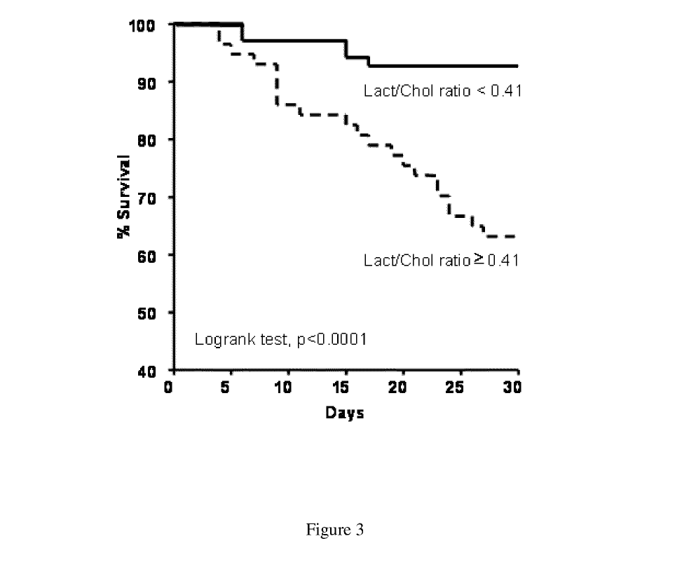

Regarding the inverse relation of lactate and cholesterol concentrations to prognosis we combined their predictive weight as the lactate/cholesterol ratio. Indeed, the lactate/cholesterol ratio was a highly accurate parameter to prognosticate the in-hospital death with 0.82 AUC with a Youden index maximum at a 0.41 lactate/cholesterol ratio. Survival analysis for 2 subgroups generated with a ratio lactate/cholesterol ratio cut-off value of 0.41 is presented under a Kaplan-Meier plot in FIG. 3. The two survival curves differed significantly (p<0.0001) which confirmed the high prognostic power for in-hospital death of the lactate/cholesterol ratio. Thus, there was a six-fold greater death risk for patients with a plasma lactate/cholesterol and a high survival rate (93%) for patients with a lactate/cholesterol value above and below 0.41, respectively.

1. Method for evaluating the vital prognosis of a subject in a critical condition, said method comprising the steps of:
i) indirectly quantifying cholesterol in a biological sample from said subject by cleaving cholesterol esters in said sample with cholesterol esterase to form cholesterol;
converting cholesterol formed in said cleaving step to cholestenone with cholesterol oxidase, and
measuring hydrogen peroxide formed in said converting step;
ii) indirectly quantifying lactate in said biological sample by oxidizing lactate with lactate oxidase, and
measuring hydrogen peroxide formed in said oxidizing step;
iii) determining, using quantities of cholesterol and lactate determined in said steps of indirectly quantifying cholesterol and indirectly quantifying lactate, a lactate/cholesterol ratio in said biological sample of said subject; and
iv) comparing the lactate/cholesterol ratio to a threshold value.

2. The method according to claim 1, wherein said biological sample is blood.

3. The method according to claim 1, wherein said biological sample is venous blood.

4. The method according to claim 1, wherein said threshold value is the mean lactate/cholesterol ratio of a population of healthy individuals.

5. The method according to claim 1, wherein said critical condition is a heart failure selected from the group consisting of chronic heart failure, an acute heart failure, a myocardial infarction, an unstable angina, a cardiac thrombus, a diastolic dysfunction and a systolic dysfunction.

6. The method according to claim 5, wherein said subject is diagnosed with comorbid conditions.

7. The method according to claim 1, wherein said critical condition is selected from the group consisting of a sepsis condition, acute breath decompensation and cardiac arrest.

8. The method according to claim 1, wherein the lactate/cholesterol ratio is obtained with concentrations of lactate and cholesterol expressed in mmol of liter of biological sample and said threshold value is between about 0.2 and about 0.7.

9. The method of claim 6, wherein said comorbid conditions are selected from the group consisting of hypertension, coronary heart disease, and diabetes mellitus.

10. The method of claim 8, wherein said threshold value is between about 0.3 and about 0.6.

11. The method of claim 8, wherein said threshold value is between about 0.3 and about 0.5.

12. The method of claim 8, wherein said threshold value is between about 0.35 and about 0.45.

13. The method of claim 8, wherein said threshold value is between about 0.37 and about 0.43.

14. The method of claim 8, wherein said threshold value is between about 0.39 and about 0.42.

15. The method of claim 8, wherein said threshold value is between about 0.40 and about 0.42.

16. The method of claim 8, wherein said threshold value is about 0.41.

17. The method of claim 1, wherein said steps of measuring hydrogen peroxide are performed using the Tinder reaction.

18. The method of claim 7, wherein said sepsis condition is septic shock.
